(19) United States
(12) Patent Application Publication
(10) Pub. No.: US 2026/0076606 A1
(43) Pub. Date: Mar. 19, 2026

(54) ECG-BASED HEARING DEVICE AND METHODS FOR OBTAINING THE ECG

(71) Applicant: Starkey Laboratories, Inc., Eden Prairie, MN (US)

(72) Inventors: Mark J. Schroeder, Eden Prairie, MN (US); Andy Shih-Hsuan Lin, Chanhassen, MN (US)

(21) Appl. No.: 19/323,372

(22) Filed: Sep. 9, 2025

Related U.S. Application Data
(60) Provisional application No. 63/695,728, filed on Sep. 17, 2024.

(51) Int. Cl. A61B 5/364; A61B 5/00; A61B 5/282; A61B 5/332; A61B 5/339; H04R 25/00; H04R 25/02

(52) U.S. Cl. CPC A61B 5/364; A61B 5/0006; A61B 5/002; A61B 5/282; A61B 5/332; A61B 5/339; A61B 5/6803; H04R 25/02; H04R 25/554; H04R 25/609; H04R 2225/021; H04R 2225/025; H04R 2225/55

(57) ABSTRACT

Disclosed herein, among other things, are systems and methods for obtaining electrocardiograms (ECGs) using hearing devices. Various aspects include a system for obtaining ECGs using hearing devices. The system includes a plurality of electrodes on or in a housing of a hearing device, and at least one processor configured to: provide instructions to a user to touch at least two of the plurality of electrodes, receive a potential measured by the plurality of electrodes, process the received potential to obtain an electrocardiogram (ECG) of the user, and output the ECG for the user on a display of a user device.

### CLAIM OF PRIORITY AND INCORPORATION BY REFERENCE

The present application claims the benefit under 35 U.S. C. § 119(e) of U.S. Provisional Patent Application 63/695,728, filed Sep. 17, 2024, the disclosure of which is hereby incorporated by reference herein in its entirety.

### TECHNICAL FIELD

This document relates generally to audio and medical device systems, and more particularly to systems and methods for obtaining electrocardiograms (ECGs) using hearing devices.

### BACKGROUND

Audio devices can be used to provide audible output to a user based on received wireless signals. Examples of audio devices include speakers and ear-wearable devices, also referred to herein as hearing devices. Example of hearing devices include hearing assistance devices or hearing instruments, including both prescriptive devices and non-prescriptive devices. Specific examples of hearing devices include, but are not limited to, hearing aids, headphones, and earbuds.

Hearing aids are used to assist patients suffering hearing loss by transmitting amplified sounds to ear canals. In one example, a hearing aid is worn in and/or around a patient's ear. Hearing aids may include processors and electronics that improve the listening experience for a specific wearer or in a specific acoustic environment.

An ECG is used to monitor a patient's heart rhythm information, and may be used to identify or detect cardiac arrythmias and abnormalities. Currently, a patient's ECG is usually only obtained by trained clinicians mostly in an infrequent in-person clinic visit. There are very limited available repeatable and reliable methods to obtain regular ECGs outside of the clinic setting. Such a method would assist in detection of early symptoms of heart disease, most commonly found in elderly patients who may use hearing aids.

Thus, there is a need in the art for improved systems and methods for obtaining ECGs.

### SUMMARY

Disclosed herein, among other things, are systems and methods for obtaining electrocardiograms (ECGs) using hearing devices. Various aspects include a system for obtaining ECGs using hearing devices. The system includes a plurality of electrodes on or in a housing of a hearing device, and at least one processor configured to: provide instructions to a user to touch at least two of the plurality of electrodes, receive a potential measured by the plurality of electrodes, process the received potential to obtain an electrocardiogram (ECG) of the user, and output the ECG for the user on a display of a user device.

Various aspects include a method for obtaining electrocardiograms (ECGs) using hearing devices. The method includes providing instructions to a user to touch at least two of a plurality of electrodes, the plurality of electrodes on or in a housing of a hearing device configured to be worn on or in an ear of the user. The method further includes receiving a potential measured by the electrodes, processing the received potential to obtain an electrocardiogram (ECG) of the user, and outputting the ECG for the user on a display of a user device.

Various aspects include a hearing device including a microphone configured to receive an input signal and provide an audio signal based on the input signal, at least two electrodes on or in a housing of the hearing device, and a processor. The processor is configured to process the audio signal to play for a wearer, provide instructions to the wearer to touch at least two of the electrodes, receive a potential measured by the electrodes, process the received potential to obtain an electrocardiogram (ECG) of the wearer, and output the ECG for the wearer on an interactive display of a computer device.

This Summary is an overview of some of the teachings of the present application and not intended to be an exclusive or exhaustive treatment of the present subject matter. Further details about the present subject matter are found in the detailed description and appended claims.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates a block diagram of a hearing device system for obtaining electrocardiograms (ECGs), according to various examples of the present subject matter.

FIGS. 2A-2B illustrate a flow diagram of method for obtaining ECGs using hearing devices, according to various examples of the present subject matter.

FIG. 2C illustrates a schematic showing a user of a hearing device system for obtaining ECGs, according to various examples of the present subject matter.

FIG. 3A illustrates a perspective view of a pair of hearing devices including electrodes for obtaining ECGs, according to various examples of the present subject matter.

FIG. 3B illustrates a side view of a hearing device including a stainless steel electrode for obtaining ECGs, according to various examples of the present subject matter.

FIG. 4A illustrates a rear view of a hearing device including multiple electrodes being used between fingers of a user, according to various examples of the present subject matter.

FIG. 4B illustrates a graphical diagram of an ECG waveform from the hearing device being used in FIG. 4A.

FIG. 4C illustrates a rear view of a hearing device including multiple electrodes being used between a finger and the head of a user, according to various examples of the present subject matter.

FIG. 4D illustrates a graphical diagram of an ECG waveform from the hearing device being used in FIG. 4C.

FIG. 5 illustrates a block diagram of a hearing device circuit, according to various examples of the present subject matter.

FIG. 6A illustrates a flow diagram of a method for obtaining ECGs using hearing devices, according to various examples of the present subject matter.

FIG. 6B illustrates a flow diagram of a method for obtaining ECGs using hearing devices and a smartphone application, according to various examples of the present subject matter.

FIG. 7 illustrates a block diagram of an example machine upon which any one or more of the techniques discussed herein may perform.

### DETAILED DESCRIPTION

The following detailed description of the present subject matter refers to subject matter in the accompanying drawings which show, by way of illustration, specific aspects and embodiments in which the present subject matter may be practiced. These embodiments are described in sufficient detail to enable those skilled in the art to practice the present subject matter. References to “an”, “one”, or “various” embodiments in this disclosure are not necessarily to the same embodiment, and such references contemplate more than one embodiment, including combinations of such embodiments. The following detailed description is demonstrative and not to be taken in a limiting sense. The scope of the present subject matter is defined by the appended claims, along with the full scope of legal equivalents to which such claims are entitled.

The present detailed description will discuss audio devices such as hearing devices and speakers. The description refers to ear-wearable devices, also referred to herein as hearing devices, which include earbuds, headsets, headphones, and hearing assistance devices using the example of hearing aids. A hearing aid may process audio signals to correct for hearing impairment of a wearer. Other hearing devices include, but are not limited to, those in this document. It is understood that their use in the description is intended to demonstrate the present subject matter, but not in a limited or exclusive or exhaustive sense.

An electrocardiograms (ECG) is used to monitor a patient's heart rhythm information, and may be used to identify or detect cardiac arrythmias and abnormalities. Currently, a patient's ECG is usually only obtained by trained clinicians mostly in an infrequent in-person clinic visit. The present subject matter provides systems and methods for obtaining ECGs using hearing devices.

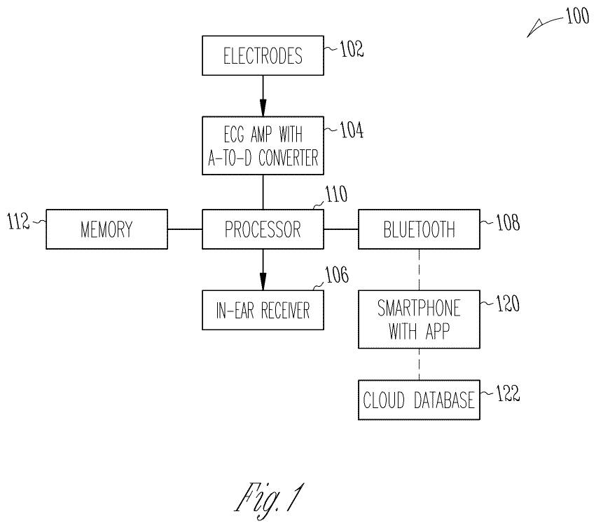

FIG. 1 illustrates a block diagram of a hearing device system 100 for obtaining electrocardiograms (ECGs), according to various examples of the present subject matter. The system 100 includes one or more electrodes 102 configured to sense heart rhythm data from a patient or wearer. In some examples, the one or more electrodes include at least two electrodes. The at least two electrodes may be integrated into a housing of a hearing device configured to be worn on or in an ear of a user, or the electrodes may be on the housing, in the housing, remote from the housing, or any combination thereof, in various examples. In some examples, one electrode is positioned to contact the ear or head of the user and another electrode is configured to be contacted by a finger of the user. Other positions or contacts of the electrodes may be used without departing from the scope of the subject matter. The system 100 also includes an ECG amplifier 104 with an analog-to-digital converter that receives signals from the electrodes 102. The ECG amplifier 104 may be within the hearing device housing, or remote from the hearing device, in various examples.

The system 100 further includes a processor 110 in communication with the ECG amplifier 104. The processor 110 may be within the housing, in various examples. The processor 110 is connected to a memory 112, a wireless communication circuit 108, and an in-ear speaker or receiver 106, in various examples. The wireless communication circuit 108 may be a Bluetooth transceiver, but other types of wireless communication circuits and protocols may be used without departing from the scope of the present subject matter. In various examples, the processor may be configured to receive signals from the at least two electrodes and/or the ECG amplifier 104, process the received signals to generate ECG data indicative of cardiac activity of the user, and analyze the ECG data to detect cardiac arrhythmias. The wireless communication circuit 108 may include a transmitter configured to transmit the ECG data to an external device, such as a smartphone 120 or a cloud database 122, in various examples.

According to various examples, the system includes three electrodes, and the processor is further configured to simultaneously acquire traditional three-lead ECG data. The electrodes 102 include one or more of conductive electrodes or capacitive electrodes. In various examples the memory 112 may include instructions for execution by the processor. In some examples, the memory 112 is configured to store the ECG data. In various examples, the hearing device of the system 100 is configured to provide instructions to the user through an associated smartphone application for positioning the electrodes 102 to achieve different ECG lead configurations. The hearing device is configured to operate in multiple modes based on the location of ECG acquisition selected by the user, including an in-ear mode and a hand-to-hand mode, in some examples. The in-ear speaker or receiver 106 functions as an audio output component configured to provide auditory feedback to the user regarding the quality of the ECG signal and instructions for improving signal quality, in various examples.

A multiple lead ECG-based hearing device and methodology for obtaining the ECG are provided. The hearing device allows a user to obtain and record their ECG from a hearing device with dry electrodes, in some examples. In various examples, two or three electrodes are placed on (such as when using a conductive electrode) or inside (such as when using a capacitive electrode) a housing of a hearing device, such as a hearing aid. The hearing device may be a behind-the-ear (BTE), in-the-ear (ITE), in-the-canal (ITC), receiver-in-canal (RIC) hearing aid, or other type of hearing aid. By using the electrodes with ECG processing components located either in or remote from the hearing device housing, an individual may acquire their ECG while either wearing or holding their hearing device.

In various examples, while wearing the hearing devices, a user may achieve up to four different ECG leads, also referred to as voltage potential vectors. These voltage potential vectors include two ipsilateral leads (left finger to left-ear hearing aid and right finger to right-ear hearing aid) and two contralateral leads (left finger to right-ear hearing aid and right finger to left-ear hearing aid). The leads may provide similar or different information than conventional leads. While ECG data may be captured simultaneously from the two ipsilateral leads, ECG data from the contralateral leads may be captured separately from the ipsilateral leads.

In addition to the four described leads, a fifth lead may be achieved by holding a hearing aid and touching the electrodes with a left and right finger simultaneously. This configuration provides the traditional Lead I ECG. Another lead may be achieved by holding an electrode of a hearing device with the left or right hand and placing the other electrode of the same hearing device on a leg of the user. Other leads or vectors may be achieved using the electrodes of the hearing device without departing from the scope of this subject matter. In one example, a three electrode hearing device is provided. Having three electrodes on the hearing device enables the user to acquire a traditional three lead ECG plus the three additional augmented leads simultaneously, in various examples. Additional numbers of electrodes on or in the device may be used without departing from the scope of the subject matter.

In some examples, the ECG data obtained using the electrodes of the hearing device may be stored and processed to determine the user's heart rate and, potentially, cardiac arrythmias and abnormalities. This provides the user with valuable information regarding the presence of potentially life-threatening heart arrythmia, e.g., atrial fibrillation, without requiring a doctor visit. Additional benefits include the convenience of having the ECG electrodes and circuitry for obtaining and/or storing ECG data it in a hearing device, which benefits the senior demographic that uses hearing assistance devices and may be most in need of heart rhythm information. In addition, placement of the electrodes on the device is done such that the electrodes cannot be contacted while the hearing device is in a charger in conjunction with either wireless charging or a charger containing an isolation transformer. In some examples, the hearing device is designed and manufactured for ease of assembly, comfort, reduced weight, and to be easier to use on the head or other body parts. The ECG process may be executed completely using only one hearing device, thus reducing the cost to the consumer.

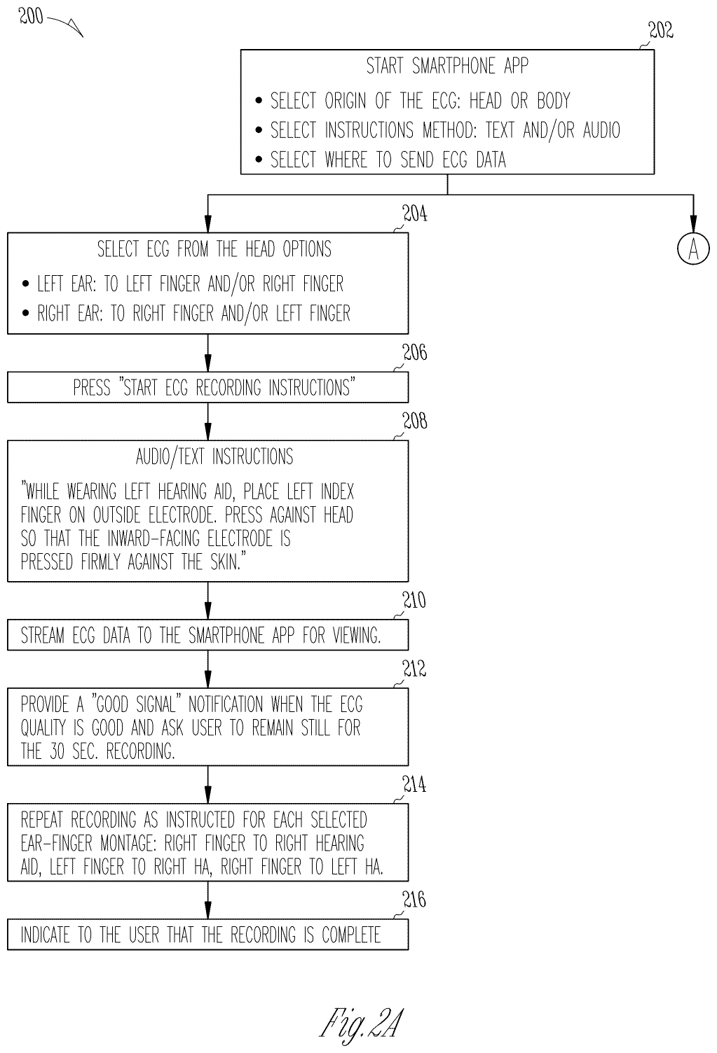

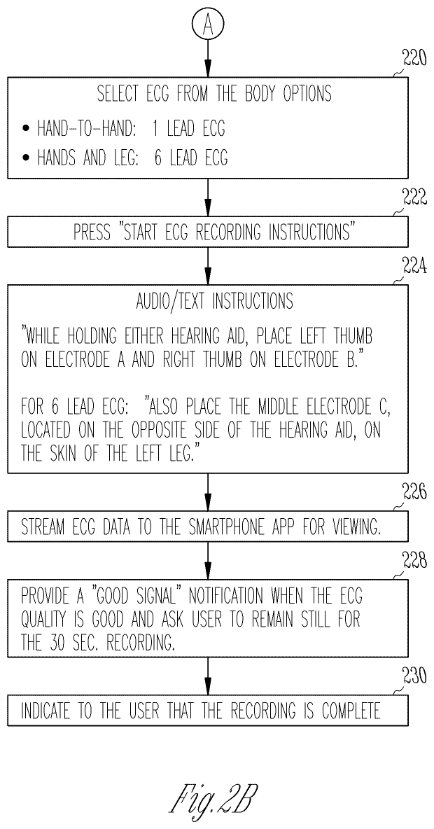

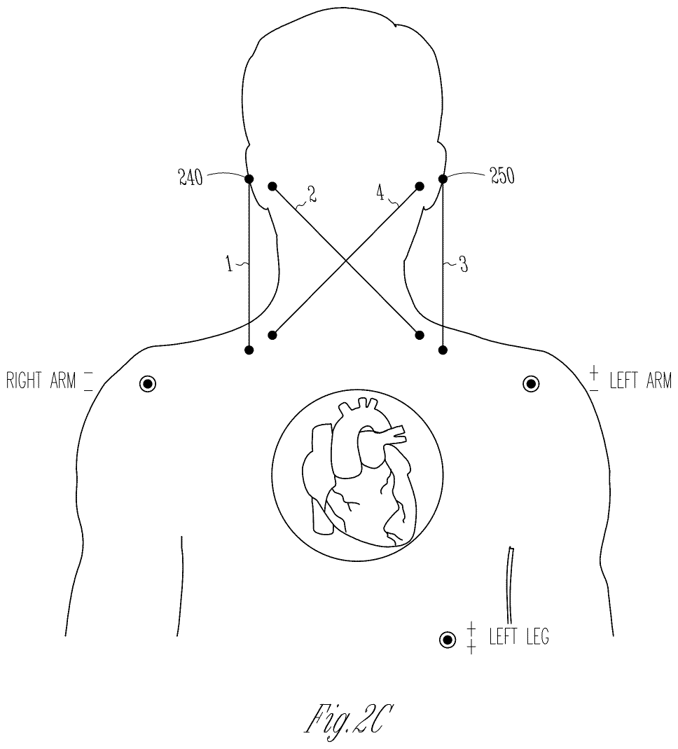

In still further examples, the electrodes may be placed on a hearing device that is designed to be placed fully in an ear such as on an in-the-ear (ITE), in-the-canal (ITC) hearing device or a RIC ear bud. In another example, capacitive electrodes are placed inside the hearing device instead of or in addition to conductive electrodes on the housing. For internal capacitive electrodes, the ECG may be obtained using any of the methods above, but off-the-ear use of the device may be preferrable, such as between the fingers or other parts of the body, to ensure adequate signal-to-noise ratio of the resulting ECG signal. In yet another example, the electrodes and processor may be built into a hearing device charger, and the user instructed to hold the charger in a desired manner to obtain the ECG data. In some examples, the ECG data may be obtained from the left ear to the left or right hand. In other examples, the ECG data may be obtained from the right ear to the left or right hand. The resulting vectors from obtaining the ECG data using these configurations are shown in FIG. 2C.

FIGS. 2A-2B illustrate a flow diagram of method for obtaining ECGs using hearing devices, according to various examples of the present subject matter. The method 200 for acquiring electrocardiogram (ECG) data using a hearing device includes a user starting a smartphone application associated with the hearing device, at step 202. The user may select the origin of the ECG through the smartphone application. The origin options include acquiring ECG from the head or from the body, in various examples. In some examples, the user may also select a method of providing instructions to the user using the smartphone application. The options may include text instructions, audio instructions, or both. The user may also select where to send the ECG data using the smartphone application, such as to the smartphone or another device.

In one example, if ECG from the head is selected, the user is presented with options for ECG acquisition at step 204. These options may include left-ear to left finger and/or right finger and right-ear to right finger and/or left finger, in some examples. At step 206, the ECG recording instructions are initiated by pressing a “Start ECG Recording Instructions” button. At step 208, audio and/or text instructions are provided to the user, for example: “While wearing left hearing aid, place left index finger on outside electrode. Press against head so that the inward-facing electrode is pressed firmly against the skin.” At step 210, ECG data is streamed to the smartphone application for viewing. At step 212, a “Good Signal” notification is provided when the ECG quality is satisfactory and instructing the user to remain still for the 30-second recording. At step 214, for the head ECG, the recording process is repeated as instructed for each selected ear-finger position, for example: right finger to right hearing device, left finger to right hearing device, and right finger to left hearing device. At step 216, an indication is provided to the user that the recording is complete.

In another example, if ECG from the body is selected, the user is presented with options for ECG acquisition at step 220. These options may include a hand-to-hand one lead ECG and a hands and leg six lead ECG, in some examples. At step 222, ECG recording instructions are initiated by pressing a “Start ECG Recording Instructions” button. At step 224, audio and/or text instructions are provided to the user, for example: “While holding either hearing aid, place left thumb on electrode A and right thumb on electrode B.” If a six lead ECG has been selected, additional instructions may be provided, for example: “Also place the middle electrode C, located on the opposite side of the hearing aid, on the skin of the left leg.” At step 226, ECG data is streamed to the smartphone application for viewing. At step 228, a “Good Signal” notification is provided when the ECG quality is satisfactory and instructing the user to remain still for the 30-second recording. At step 230, an indication is provided to the user that the recording is complete. In various examples, the method 200 provides for versatile ECG data acquisition using a hearing aid device, with user guidance provided through a smartphone application interface. In some examples, the device may output the ECG from the wearer on an interactive display of a computer device or mobile device.

While the above examples refer to using a smartphone application, the present system may be used without such an application. For example, a user may press on the side of the hearing device to initiate the ECG, and instructions to the user may be provided via audio directly to the ear using the hearing device. In some examples, a mix of smartphone application and ear pressing and audio may be used to direct the user to perform the steps for obtaining the ECG data. In one example, the user may use a predetermined pattern of taps on the device, such a double tap, to initiate the sequence of measuring all vectors for the ECG. In further examples, the present system provides the ability for the user to trigger the ECG data collection by touching the electrode on the hearing device. In this manner, if the user feels that they are having a heart issue or feeling unhealthy, the present system provides the ability to quickly collect the ECG data in response to the user triggering the collection, which may be very insightful for diagnosis. In some examples, the software application may provide a notification to the user that the ECG data was successfully collected. The software may also prompt the user to determine whether the user would like to follow up with data collection of one or more additional ECG lead configurations, the software application including instructions to the user to obtain the additional ECG data. In addition, if the user declines the follow up data collection, the system may categorize the initial electrode configuration using the ECG waveform morphology, and the application may also prompt the user to confirm this electrode configuration, in some examples.

FIG. 2C illustrates a schematic showing a user of a hearing device system for obtaining ECGs, according to various examples of the present subject matter. A number of different user positions are demonstrated using hearing devices in the users ear. In one position 1 (or vector 1), the user places a right finger on an electrode of a right-ear hearing device 240 to obtain the ECG data. In another position 2 (or vector 2), the user places a left finger on an electrode of a left-ear hearing device 250 to obtain the ECG data. In yet another position 3 (or vector 3), the user places a left finger on an electrode of a right-ear hearing device 240 to obtain the ECG data. In a further position 4 (or vector 4), the user places a right finger on an electrode of a left-ear hearing device 250 to obtain the ECG data. Other positions are possible without departing from the subject matter. For example, the user may take one or more of the right-ear hearing device 240 or the left-ear hearing device 250 and hold the device between fingers of opposite hands to obtain the ECG data. Or a user may press one or more of the right-ear hearing device 240 or the left-ear hearing device 250 against another body part to obtain the ECG data. The objective with placement of the electrodes is to obtain a vector from the left side of the body to the right side of the body to obtain the ECG signals across the user's heart, in various examples.

In some examples, the ECG data may be recorded while the hearing device is on the left ear of the user by touching the outward facing electrode with a left-hand finger or with a right-hand finger. Likewise, when a hearing device is placed on the right ear, the outer electrode can be contacted with either hand. These four cases may provide slightly different voltage potential vectors, or viewing angles, of the electrical activity of the heart, as shown in FIG. 2C.

In one example, a hearing device electrode may be placed on the user's chest while a finger is placed on the hearing device's other electrode to obtain a lead vector. Similarly, a user may touch the hearing device electrodes on opposite sides of the hearing device with both index fingers (while the device is off the head) to obtain the ECG across the chest and heart.

The hearing device may begin logging ECG data upon an indication of receiving data from the electrodes. In some examples, the logged ECG data may only be analyzed for heart conditions if the duration of the logged data meets a minimum programmable threshold, such as 30 seconds or 1 minute, to remove non-consistent data. In various examples, by clearly defining and controlling the user movement to specific electrode placement and touching, the heart condition assessment is more accurate and reliable.

In various examples, once the user electrode placement action is confirmed and the ECG data has been recorded or logged, the present subject matter performs an analysis or calculation using the logged data to detect the presence or onset of a heart condition. For example, the logged data can be assessed based on the frequency, amplitude, duration, or timing of the heart rhythm of the user. In some examples, the logged data is compared to previous data from the user, from other users or from a known ECG waveform database. Other algorithms for heart condition detection may be used without departing from the scope of the present subject matter. In addition, various algorithms may be used to predict cardiac arrhythmias or other heart conditions using the logged sensor data by the system.

In various examples, the system may provide an audio prompt to the user using a hearing device in the other ear of the user (that has yet to be removed) that informs the user that the data is being logged. For example, the prompt may indicate that “ECG measurement starting” to alert the user to the recording and focus them on the task of proper electrode placement. In various examples, the hearing device may communicate with another device, such as a smart phone or other wireless device, to log the data, process the data, and/or provide notifications to the user.

The present subject matter provides a plurality of benefits and improvements for medical diagnostics. For example, the present system identifies symptoms of heart conditions in a population of hearing device users that is typically of advanced age. The present system uses electrodes present on or in hearing devices, thus reducing or eliminating additional costs of implementation. In some examples, the progression of worsening of ECG data may be recorded, and the progress tracked and used in the medical evaluation of the user.

According to various examples, the present system processes collected data using a processor in or on the hearing device. Other processors may be used, such as in remote devices (e.g., in a smartphone, cloud, etc.) that are in communication with the hearing device. The logged data may be stored locally on the hearing device or remotely, such as on cloud storage, or using an application on mobile device. In some examples, the present system uses machine learning, including but not limited to a deep neural network or artificial intelligence, to process the tracked data. The machine learning processor, either local or remote, may be trained on data from the user and/or data from multiple users or participants, to identify heart conditions based on the ECG, including identifying ECG patterns that may be indicative of heart conditions for individuals and for the general population.

The system provides notifications of heart conditions or ECG irregularities, in various examples. In some examples, the system provides notifications to the user via the hearing device or in a smartphone application associated with the hearing device. The system may also or alternatively provide notifications to a healthcare provider of the user, such that the user's doctor or other individual (such as another health care provider, family member, or the like) may also keep track of patient data, symptoms, and/or heart condition identification or determination.

In one example, the ECG data is logged in a hearing device. The ECG data may be logged in external devices or in the cloud, in various examples. In various examples, the present method and system may be programmed into firmware of the device. In other examples, a separate program or device may be used to track and record the ECG data for the user.

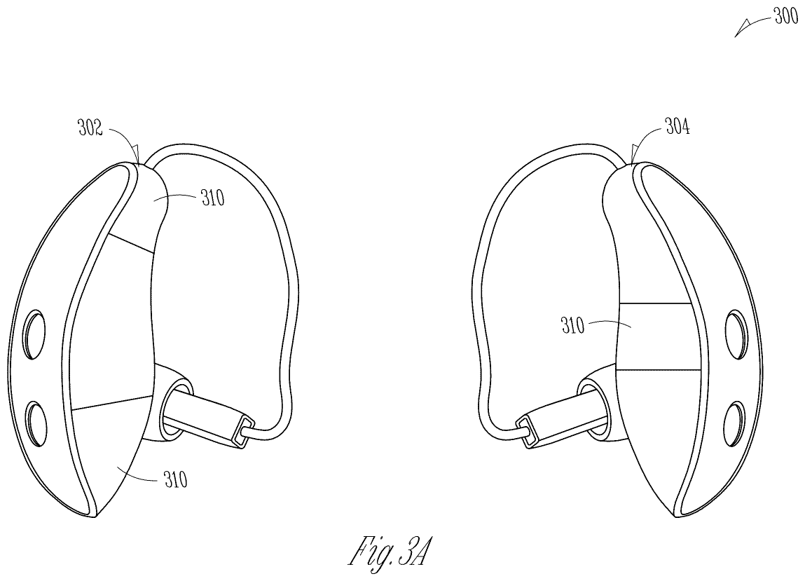

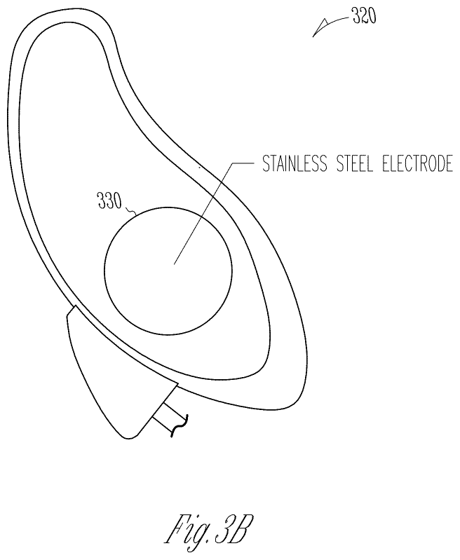

FIG. 3A illustrates a perspective view 300 of two different embodiments of hearing devices 302, 304 including electrodes for obtaining ECGs, according to various examples of the present subject matter. In one embodiment, the hearing device 302 includes two electrodes 310 on one side of the hearing device plus an additional electrode on the opposite side (not shown) of the hearing device 302. In another embodiment, a hearing device 304 includes one electrode 310 plus an additional electrode on the opposite side (not shown) of the hearing device 304. The hearing device 302 with three electrodes may be used to obtain a six lead ECG, in some examples. The hearing device 304 with two electrodes may be used to obtain one ECG lead at a time, in another example.

FIG. 3B illustrates a side view of a hearing device 320 including a stainless steel electrode 330 for obtaining ECGs, according to various examples of the present subject matter. Multiple stainless steel electrodes 330 may be included on various surfaces of the hearing device 320, in some examples. Although three electrodes might provide a slightly better signal, it may be preferable to use only two electrodes for ease of manufacture, as the processor and electronics enable the ECG to be easily acquired with just two electrodes while still providing a usable ECG waveform. The electrode 330 may be built into a hearing device shell or housing, in some examples. In FIG. 3B, a second electrode is located on the opposite side of the device housing (not shown). In some examples, the electrode or electrodes may cover a predetermined amount of surface area of the device housing. In one example, the electrode or electrodes may cover twenty percent of the surface area of the device housing.

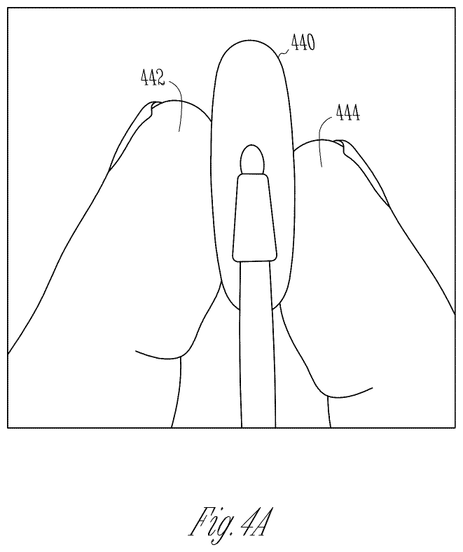

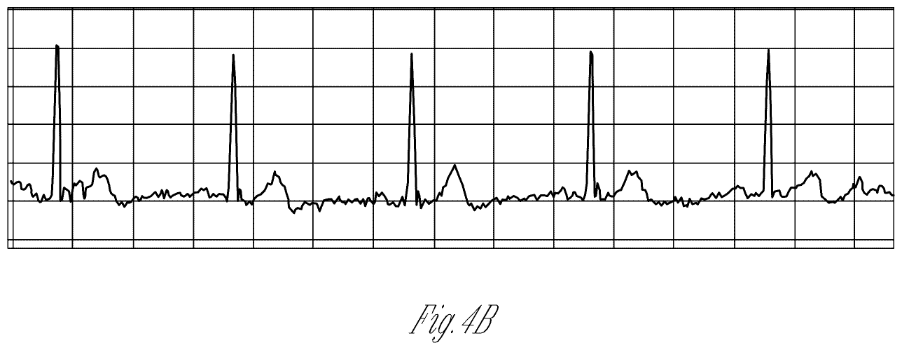

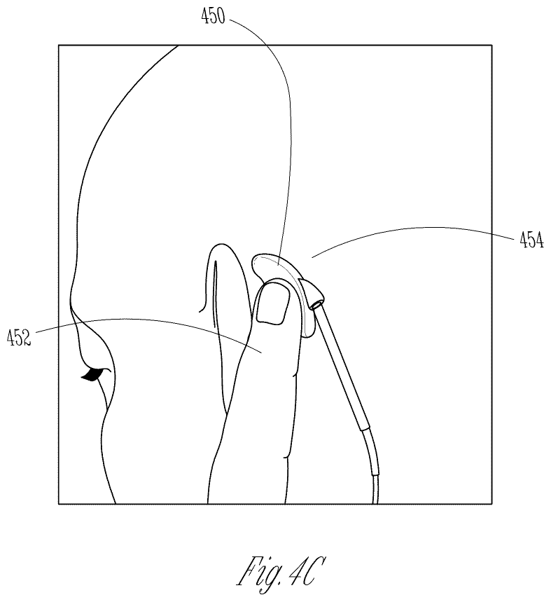

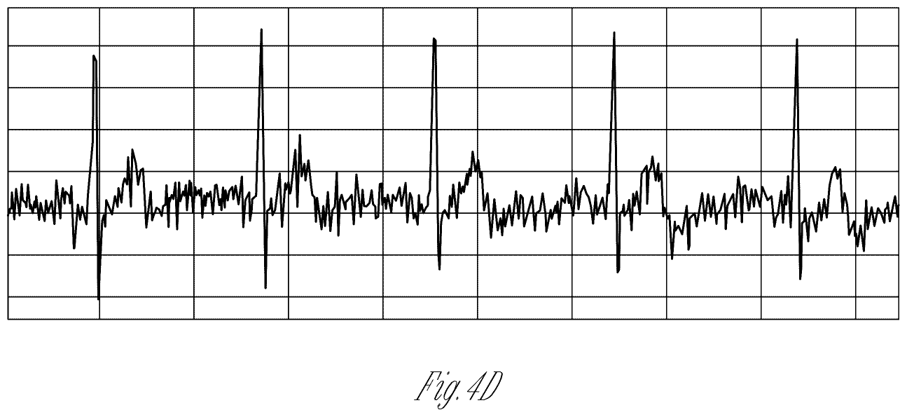

FIG. 4A illustrates a rear view of a hearing device 440 including multiple electrodes being used between fingers of a user, according to various examples of the present subject matter. In this example, a user holds the hearing device 440 between a left index finger 442 and a right index finger 444. The left index finger 442 contacts an electrode on the left side of the hearing device 440 and the right index finger 444 contacts an electrode on the right side of the hearing device 440 to obtain the ECG data. FIG. 4B illustrates a graphical diagram of an ECG waveform from the hearing device being used in FIG. 4A.

FIG. 4C illustrates a rear view of a hearing device 450 including multiple electrodes being used between a finger and the head of a user, according to various examples of the present subject matter. In this example, a user presses the hearing device 450 between a left index finger 452 and the left side of the head 454 of the user. The left index finger 452 contacts an electrode on the left side of the hearing device 450 and the left side of the head 454 contacts an electrode on the right side of the hearing device 450 to obtain the ECG data. FIG. 4D illustrates a graphical diagram of an ECG waveform from the hearing device being used in FIG. 4C.

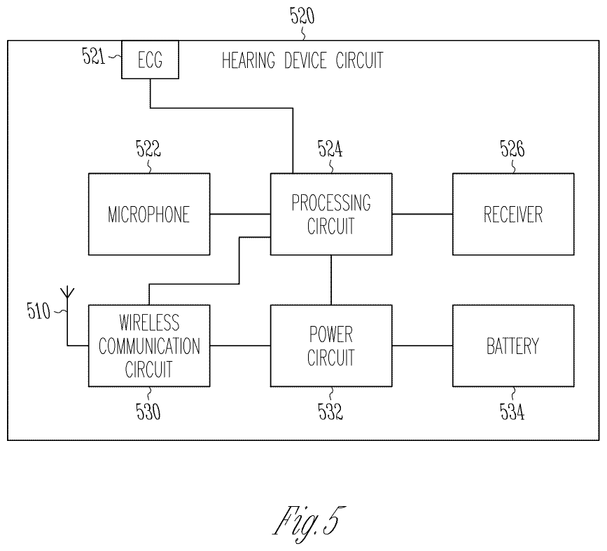

FIG. 5 illustrates a block diagram of a hearing device circuit, according to various examples of the present subject matter. Hearing device circuit 520 represents an example of portions of a hearing device and includes a microphone 522, a wireless communication circuit 530, an antenna 510, an electrocardiogram (ECG) circuit 521 including or connected to electrodes (not shown), a processing circuit 524, a receiver (speaker) 526, a battery 534, and a power circuit 532. Microphone 522 receives sounds from the environment of the hearing device user (wearer of the hearing device). Wireless communication circuit 530 communicates with another device wirelessly using antenna 510, including receiving programming codes, streamed audio signals, and/or other audio signals and transmitting programming codes, audio signals, and/or other signals. Examples of the other device includes other hearing devices of other users, another hearing device of a pair of hearing devices for the same wearer, a hearing device host device, an assistive listening device (ALD), an audio streaming device, a smartphone, and other devices capable of communicating with hearing devices wirelessly. Processing circuit 524 controls the operation of a hearing device using the programming codes and processes the sounds received by microphone 522 and/or the audio signals received by wireless communication circuit 530 to produce output sounds. Receiver 526 transmits output sounds to an ear canal of the hearing device wearer. Battery 534 and power circuit 532 constitute the power source for the operation of hearing device circuit 520. In one example, power circuit 532 can include a power management circuit. In another alternative or additional example, battery 534 can include a rechargeable battery and power circuit 532 can include a recharging circuit for recharging the rechargeable battery.

In various examples, the hearing device is configured for obtaining ECGs from a patient or wearer. The ECG circuit 521 may include two or more electrodes for obtaining heart beat signals from the patient or wearer, in various examples. In one example, the ECG circuit 521 includes at least two electrodes. While the ECG circuit 521 is depicted within the hearing device, the ECG circuit 521 or portions thereof may be outside the device, incorporated into a housing of the device, or in any other position inside or outside the device. The hearing device circuit 520 includes at least one processor or processing circuit 524 and data storage in communication with the processing circuit 524. The data storage comprises instructions thereon that, when executed by the processing circuit 524, causes the processing circuit 524 to perform the functions of the present systems and methods. For example, the processing circuit may perform operations to: receive signals from the at least two electrodes of the ECG circuit 521 or electrodes connected to the ECG circuit 521, process the received signals to generate ECG data indicative of cardiac activity of the user, and analyze the ECG data to detect cardiac arrhythmias. The wireless communication circuit 530 may include a transmitter configured to transmit the ECG data to an external device. While the ECG circuit 521 is depicted within the hearing device, the ECG circuit 521 may be outside the device, incorporated into a housing of the device, or in any other position inside or outside the device. The hearing device circuit 520 may be included in an ear bud, headphones, a hearing aid, or other ear-wearable device, in various examples.

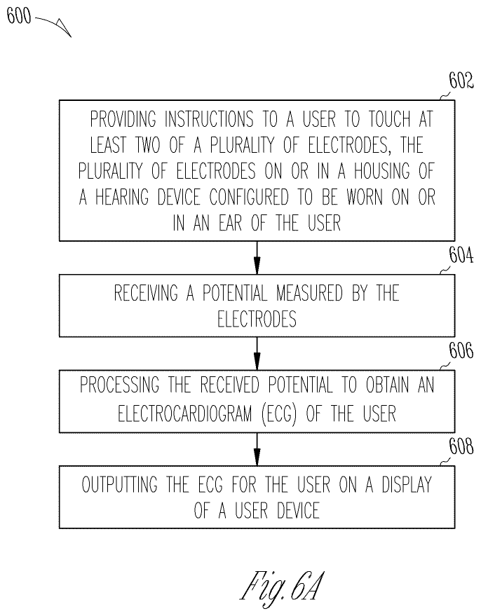

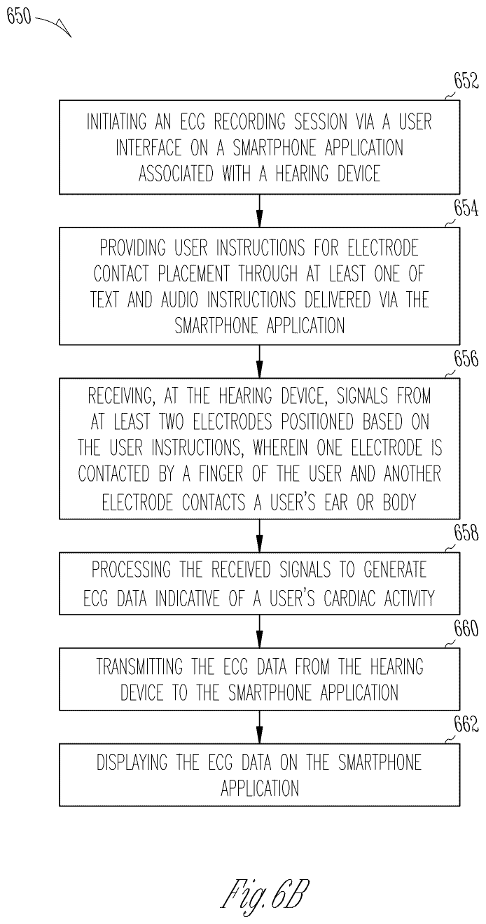

FIG. 6A illustrates a flow diagram of a method for obtaining ECGs using hearing devices, according to various examples of the present subject matter. At step 602, the method 600 includes providing instructions to a user to touch at least two of a plurality of electrodes, the plurality of electrodes on or in a housing of a hearing device configured to be worn on or in an ear of the user. In various examples, the user may be instructed to touch the at least two electrodes with a finger, their head (by pushing on the opposite side of the device with a finger), their leg, or some combination of these or other body parts. The method 600 further includes receiving a potential measured by the electrodes, at step 604, and processing the received potential to obtain an electrocardiogram (ECG) of the user, at step 606. At step 608, the method 600 includes outputting the ECG for the user on a display of a user device.

In various examples, the user device includes a smart phone configured to communicate with the hearing device. The user device includes a mobile computer configured to communicate with the hearing device, in some examples. In one example, the user device includes a tablet configured to communicate with the hearing device.

FIG. 6B illustrates a flow diagram of a method for acquiring electrocardiogram (ECG) data using a hearing device, according to various examples of the present subject matter. The method 650 includes initiating an ECG recording session via a user interface on a smartphone application associated with the hearing device, at step 652. At step 654, the method 650 includes providing user instructions for electrode contact placement through at least one of text and audio instructions delivered via the smartphone application. The method 650 further includes receiving, at the hearing aid device, signals from at least two electrodes positioned based on the user instructions, wherein one electrode is contacted by a finger of the user and another electrode contacts the user's ear or body, at step 656. At step 658, the received signals are processed to generate ECG data indicative of the user's cardiac activity. The ECG data is transmitted from the hearing device to the smartphone application, at step 660, and the ECG data is displayed on the smartphone application, at step 662.

According to various examples, the method 650 may further include providing feedback to the user regarding the quality of the ECG data and instructing the user to remain still if the ECG data quality is satisfactory. The method 650 may further include repeating the ECG data acquisition as necessary based on additional user selections for different electrode configurations, in some examples.

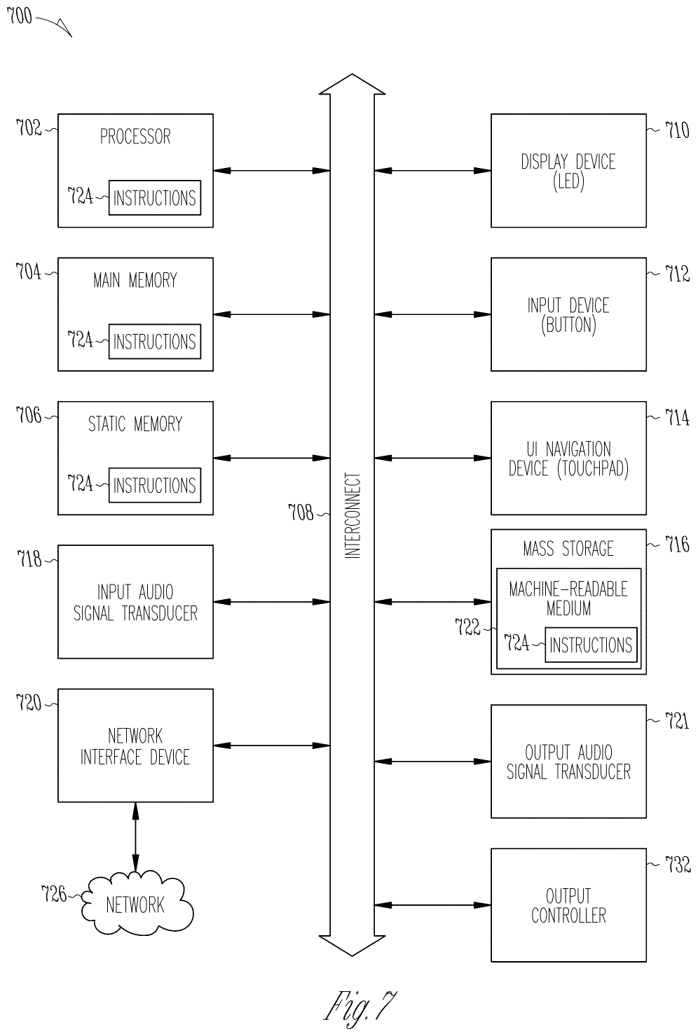

FIG. 7 illustrates a block diagram of an example machine 700 upon which any one or more of the techniques (e.g., methodologies) discussed herein may perform. In alternative examples, the machine 700 may operate as a standalone device or may be connected (e.g., networked) to other machines. In a networked deployment, the machine 700 may operate in the capacity of a server machine, a client machine, or both in server-client network environments. In an example, the machine 700 may act as a peer machine in peer-to-peer (P2P) (or other distributed) network environment. The machine 700 may be a personal computer (PC), a tablet PC, a set-top box (STB), a personal digital assistant (PDA), a mobile telephone, a hearing device, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 700 may include a hardware processor 702 (e.g., a central processing unit (CPU), a graphics processing unit (GPU), a hardware processor core, or any combination thereof), a main memory 704, and a static memory 706, some or all of which may communicate with each other via an interlink (e.g., bus) 408. The machine 700 may further include a display unit 710, an alphanumeric input device 712 (e.g., a keyboard), and a user interface (UI) navigation device 714 (e.g., a mouse). In an example, the display unit 710, input device 712, and UI navigation device 714 may be a touch screen display. The machine 700 may additionally include a storage device (e.g., drive unit) 416, one or more input audio signal transducers 718 (e.g., microphone), a network interface device 720, and one or more output audio signal transducers 721 (e.g., speaker). The machine 700 may include an output controller 732, such as a serial (e.g., universal serial bus (USB), parallel, or other wired or wireless (e.g., infrared (IR), near-field communication, etc.) connection to communicate or control one or more peripheral devices (e.g., a printer, card reader, etc.).

The storage device 716 may include a machine readable medium 722 on which is stored one or more sets of data structures or instructions 724 (e.g., software) embodying or utilized by any one or more of the techniques or functions described herein. The instructions 724 may also reside, completely or at least partially, within the main memory 704, within static memory 706, or within the hardware processor 702 during execution thereof by the machine 700. In an example, one or any combination of the hardware processor 702, the main memory 704, the static memory 706, or the storage device 716 may constitute machine readable media.

While the machine readable medium 722 is illustrated as a single medium, the term “machine readable medium” may include a single medium or multiple media (e.g., a centralized or distributed database, and/or associated caches and servers) configured to store the one or more instructions 724.

The term “machine readable medium” may include any medium that is capable of storing, encoding, or carrying instructions for execution by the machine 700 and that cause the machine 700 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine-readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EEPROM) and flash memory devices; magnetic disks, such as internal hard disks and removable disks; magneto-optical disks; and CD-ROM and DVD-ROM disks.

The instructions 724 may further be transmitted or received over a communications network 726 using a transmission medium via the network interface device 720 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 720 may include one or more physical jacks (e.g., Ethernet, coaxial, or phone jacks) or one or more antennas to connect to the communications network 726. In an example, the network interface device 720 may include a plurality of antennas to communicate wirelessly using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 700, and includes digital or analog communications signals or other intangible medium to facilitate communication of such software.

Various examples of the present subject matter support wireless communications with a hearing device. In various examples the wireless communications may include standard or nonstandard communications. Some examples of standard wireless communications include link protocols including, but not limited to, Bluetooth™, BLE, Auracast, IEEE 802.11(wireless LANs), 802.15 (WPANs), 802.16 (WiMAX), cellular protocols including, but not limited to CDMA and GSM, ZigBee, and ultra-wideband (UWB) technologies. Such protocols support radio frequency communications and some support infrared communications while others support NFMI. Although the present system is demonstrated as a radio system, it is possible that other forms of wireless communications may be used such as ultrasonic, optical, infrared, and others. It is understood that the standards which may be used include past and present standards. It is also contemplated that future versions of these standards and new future standards may be employed without departing from the scope of the present subject matter.

The wireless communications support a connection from other devices. Such connections include, but are not limited to, one or more mono or stereo connections or digital connections having link protocols including, but not limited to 802.3 (Ethernet), 802.4, 802.5, USB, SPI, PCM, ATM, Fibre-channel, Firewire or 1394, InfiniBand, or a native streaming interface. In various examples, such connections include all past and present link protocols. It is also contemplated that future versions of these protocols and new future standards may be employed without departing from the scope of the present subject matter.

Hearing assistance devices typically include at least one enclosure or housing, a microphone, hearing assistance device electronics including processing electronics, and a speaker or “receiver. ” Hearing assistance devices may include a power source, such as a battery. In various examples, the battery is rechargeable. In various examples multiple energy sources are employed. It is understood that in various examples the microphone is optional. It is understood that in various examples the receiver is optional. It is understood that variations in communications protocols, antenna configurations, and combinations of components may be employed without departing from the scope of the present subject matter. Antenna configurations may vary and may be included within an enclosure for the electronics or be external to an enclosure for the electronics. Thus, the examples set forth herein are intended to be demonstrative and not a limiting or exhaustive depiction of variations.

It is understood that digital hearing assistance devices include a processor. In digital hearing assistance devices with a processor, programmable gains may be employed to adjust the hearing assistance device output to a wearer's particular hearing impairment. The processor may be a digital signal processor (DSP), microprocessor, microcontroller, other digital logic, or combinations thereof. The processing may be done by a single processor, or may be distributed over different devices. The processing of signals referenced in this application may be performed using the processor or over different devices. Processing may be done in the digital domain, the analog domain, or combinations thereof. Processing may be done using subband processing techniques. Processing may be done using frequency domain or time domain approaches. Some processing may involve both frequency and time domain aspects. For brevity, in some examples drawings may omit certain blocks that perform frequency synthesis, frequency analysis, analog-to-digital conversion, digital-to-analog conversion, amplification, buffering, and certain types of filtering and processing. In various examples of the present subject matter the processor is adapted to perform instructions stored in one or more memories, which may or may not be explicitly shown. Various types of memory may be used, including volatile and nonvolatile forms of memory. In various examples, the processor or other processing devices execute instructions to perform a number of signal processing tasks. Such examples may include analog components in communication with the processor to perform signal processing tasks, such as sound reception by a microphone, or playing of sound using a receiver (i.e., in applications where such transducers are used). In various examples of the present subject matter, different realizations of the block diagrams, circuits, and processes set forth herein may be created by one of skill in the art without departing from the scope of the present subject matter.

It is further understood that different hearing devices may embody the present subject matter without departing from the scope of the present disclosure. The devices depicted in the figures are intended to demonstrate the subject matter, but not necessarily in a limited, exhaustive, or exclusive sense. It is also understood that the present subject matter may be used with a device designed for use in the right ear or the left ear or both ears of the wearer.

The present subject matter is demonstrated for hearing devices, including hearing assistance devices, including but not limited to, behind-the-ear (BTE), in-the-ear (ITE), in-the-canal (ITC), receiver-in-canal (RIC), invisible-in-canal (IIC) or completely-in-the-canal (CIC) type hearing assistance devices. It is understood that behind-the-ear type hearing assistance devices may include devices that reside substantially behind the ear or over the ear. Such devices may include hearing assistance devices with receivers associated with the electronics portion of the behind-the-ear device, or hearing assistance devices of the type having receivers in the ear canal of the user, including but not limited to RIC or receiver-in-the-ear (RITE) designs. The present subject matter may also be used in hearing assistance devices generally, such as cochlear implant type hearing devices. The present subject matter may also be used in deep insertion devices having a transducer, such as a receiver or microphone. The present subject matter may be used in bone conduction or otherwise osseointegrated hearing devices, in some examples. The present subject matter may be used in devices whether such devices are standard or custom fit and whether they provide an open or an occlusive design. It is understood that other hearing devices not expressly stated herein may be used in conjunction with the present subject matter.

### OTHER NOTES AND EXAMPLES

Example 1 is a system including a plurality of electrodes on or in a housing of a hearing device and at least one processor configured to: provide instructions to a user to touch at least two of the plurality of electrodes, receive a potential measured by the plurality of electrodes, process the received potential to obtain an electrocardiogram (ECG) of the user, and output the ECG for the user on a display of a user device.

In Example 2, the subject matter of Example 1 optionally includes wherein the hearing device is a hearing aid.

In Example 3, the subject matter of Example 2 optionally includes wherein the hearing aid is a behind-the-ear (BTE) hearing aid.

In Example 4, the subject matter of Example 2 optionally includes wherein the hearing aid is an in-the-ear (ITE) hearing aid.

Example 5 is a method including providing instructions to a user to touch at least two of a plurality of electrodes, the plurality of electrodes on or in a housing of a hearing device configured to be worn on or in an ear of the user, receiving a potential measured by the electrodes, processing the received potential to obtain an electrocardiogram (ECG) of the user, and outputting the ECG for the user on a display of a user device.

In Example 6, the subject matter of Example 5 optionally includes wherein the user device includes a smart phone configured to communicate with the hearing device.

In Example 7, the subject matter of Example 5 optionally includes wherein the user device includes a mobile computer configured to communicate with the hearing device.

In Example 8, the subject matter of Example 5 optionally includes wherein the user device includes a tablet configured to communicate with the hearing device.

Example 9 is a hearing device including a microphone configured to receive an input signal and provide an audio signal based on the input signal, at least two electrodes on or in a housing of the hearing device, and a processor configured to: process the audio signal to play for a wearer, provide instructions to the wearer to touch at least two of the electrodes, receive a potential measured by the electrodes, process the received potential to obtain an electrocardiogram (ECG) of the wearer, and output the ECG for the wearer on an interactive display of a computer device.

In Example 10, the subject matter of Example 9 optionally includes wherein the processor is further configured to process the audio signal to correct for hearing impairment of a wearer.

In Example 11, the subject matter of Example 9 or Example 10 optionally includes wherein the computer device includes a smart phone configured to communicate with the hearing device.

In Example 12, the subject matter of Example 9 or Example 10 optionally includes wherein the computer device includes a server configured to communicate with the hearing device.

Example 13 is a hearing device including a housing configured to be worn on or in an ear of a user, at least two electrodes integrated into the housing, wherein one electrode is positioned to contact the ear or head of the user and another electrode is configured to be contacted by a finger of the user, a processor within the housing, the processor configured to: receive signals from the at least two electrodes, process the received signals to generate ECG data indicative of cardiac activity of the user, and analyze the ECG data to detect cardiac arrhythmias, and a transmitter configured to transmit the ECG data to an external device.

In Example 14, the subject matter of Example 13 optionally includes wherein the device includes three electrodes, and the processor is further configured to simultaneously acquire traditional three-lead ECG data.

In Example 15, the subject matter of Example 13 or Example 14 optionally includes wherein the electrodes include one or more of conductive electrodes or capacitive electrodes.

In Example 16, the subject matter of any of Examples 13-15 optionally includes wherein the external device is a smartphone, and the transmitter includes a Bluetooth transmitter.

In Example 17, the subject matter of any of Examples 13-16 optionally further includes a memory configured to store the ECG data.

In Example 18, the subject matter of any of Examples 13-17 optionally includes wherein the device is configured to provide instructions to the user through an associated smartphone application for positioning the electrodes to achieve different ECG lead configurations.

In Example 19, the subject matter of any of Examples 13-18 optionally includes wherein the device is configured to operate in multiple modes based on a location of ECG acquisition selected by the user, including an in-ear mode and a hand-to-hand mode.

In Example 20, the subject matter of any of Examples 13-19 optionally further includes an audio output component configured to provide auditory feedback to the user regarding quality of the ECG signal and instructions for improving signal quality.

Example 21 is a method for acquiring electrocardiogram (ECG) data using a hearing device, the method including initiating an ECG recording session via a user interface on a smartphone application associated with the hearing device;, providing user instructions for electrode contact placement through at least one of text and audio instructions delivered via the smartphone application, receiving, at the hearing device, signals from at least two electrodes positioned based on the user instructions, wherein one electrode is contacted by a finger of the user and another electrode contacts a user's ear or body, processing the received signals to generate ECG data indicative of a user's cardiac activity, transmitting the ECG data from the hearing device to the smartphone application, and displaying the ECG data on the smartphone application.

In Example 22, the subject matter of Example 21 optionally further includes providing feedback to the user regarding quality of the ECG data and instructing the user to remain still if the ECG data quality is satisfactory.

In Example 23, the subject matter of Example 21 or Example 22 optionally further includes repeating ECG data acquisition as necessary based on additional user selections for different electrode configurations.

Example 24 is at least one machine-readable medium including instructions that, when executed by processing circuitry, cause the processing circuitry to perform operations to implement of any of Examples 1-23.

Example 25 is an apparatus comprising means to implement of any of Examples 1-23.

Example 26 is a system to implement of any of Examples 1-23.

Example 27 is a method to implement of any of Examples 1-23.

This application is intended to cover adaptations or variations of the present subject matter. It is to be understood that the above description is intended to be illustrative, and not restrictive. The scope of the present subject matter should be determined with reference to the appended claims, along with the full scope of legal equivalents to which such claims are entitled.

What is claimed is:

1. A system comprising:
a plurality of electrodes on or in a housing of a hearing device; and
at least one processor configured to:
provide instructions to a user to touch at least two of the plurality of electrodes;
receive a potential measured by the plurality of electrodes;
process the received potential to obtain an electrocardiogram (ECG) of the user; and
output the ECG for the user on a display of a user device.

2. The system of claim 1, wherein the hearing device is a hearing aid.

3. The system of claim 2, wherein the hearing aid is a behind-the-ear (BTE) hearing aid.

4. The system of claim 2, wherein the hearing aid is an in-the-ear (ITE) hearing aid.

5. A method comprising:
providing instructions to a user to touch at least two of a plurality of electrodes, the plurality of electrodes on or in a housing of a hearing device configured to be worn on or in an ear of the user;
receiving a potential measured by the electrodes;
processing the received potential to obtain an electrocardiogram (ECG) of the user; and
outputting the ECG for the user on a display of a user device.

6. The method of claim 5, wherein the user device includes a smart phone configured to communicate with the hearing device.

7. The method of claim 5, wherein the user device includes a mobile computer configured to communicate with the hearing device.

8. The method of claim 5, wherein the user device includes a tablet configured to communicate with the hearing device.

9. A hearing device comprising:
a microphone configured to receive an input signal and provide an audio signal based on the input signal;
at least two electrodes on or in a housing of the hearing device; and
a processor configured to:
process the audio signal to play for a wearer;
provide instructions to the wearer to touch at least two of the electrodes;
receive a potential measured by the electrodes;
process the received potential to obtain an electrocardiogram (ECG) of the wearer; and
output the ECG for the wearer on an interactive display of a computer device.

10. The hearing device of claim 9, wherein the processor is further configured to:
process the audio signal to correct for hearing impairment of a wearer.

11. The hearing device of claim 9, wherein the computer device includes a smart phone configured to communicate with the hearing device.

12. The hearing device of claim 9, wherein the computer device includes a server configured to communicate with the hearing device.

13. A hearing device comprising:
a housing configured to be worn on or in an ear of a user;
at least two electrodes integrated into the housing, wherein one electrode is positioned to contact the ear or head of the user and another electrode is configured to be contacted by a finger of the user;
a processor within the housing, the processor configured to:
receive signals from the at least two electrodes;
process the received signals to generate ECG data indicative of cardiac activity of the user; and
analyze the ECG data to detect cardiac arrhythmias; and
a transmitter configured to transmit the ECG data to an external device.

14. The hearing device of claim 13, wherein the device includes three electrodes, and the processor is further configured to simultaneously acquire traditional three-lead ECG data.

15. The hearing device of claim 13, wherein the electrodes include one or more of conductive electrodes or capacitive electrodes.

16. The hearing device of claim 13, wherein the external device is a smartphone, and the transmitter includes a Bluetooth transmitter.

17. The hearing device of claim 13, further comprising a memory configured to store the ECG data.

18. The hearing device of claim 13, wherein the device is configured to provide instructions to the user through an associated smartphone application for positioning the electrodes to achieve different ECG lead configurations.

19. The hearing device of claim 13, wherein the device is configured to operate in multiple modes based on a location of ECG acquisition selected by the user, including an in-ear mode and a hand-to-hand mode.

20. The hearing device of claim 13, further comprising an audio output component configured to provide auditory feedback to the user regarding quality of the ECG signal and instructions for improving signal quality.

21. A method for acquiring electrocardiogram (ECG) data using a hearing device, the method comprising:
initiating an ECG recording session via a user interface on a smartphone application associated with the hearing device;
providing user instructions for electrode contact placement through at least one of text and audio instructions delivered via the smartphone application;
receiving, at the hearing device, signals from at least two electrodes positioned based on the user instructions, wherein one electrode is contacted by a finger of the user and another electrode contacts a user's ear or body;
processing the received signals to generate ECG data indicative of a user's cardiac activity;
transmitting the ECG data from the hearing device to the smartphone application; and
displaying the ECG data on the smartphone application.

22. The method of claim 21, further comprising:
providing feedback to the user regarding quality of the ECG data and instructing the user to remain still if the ECG data quality is satisfactory.

23. The method of claim 21, further comprising:
repeating ECG data acquisition as necessary based on additional user selections for different electrode configurations.
